(19) United States
(12) Patent Application Publication
(10) Pub. No.: US 2005/0119481 A1
(43) Pub. Date: Jun. 2, 2005

(54) Novel amidine derivatives and the use of the same in pharmaceuticals

(76) Inventors: Hartmut Rehwinkel, Berlin (DE); Peter Hoelscher, Berlin (DE); Stefan Jaroch, Berlin (DE); Detlev Suelzle, Berlin (DE); Margrit Hillmann, Berlin (DE); Gerardine Anne Burton, Berlin (DE); Fiona Macdougall McDonald, Berlin (DE)

Correspondence Address: BERLEX BIOSCIENCES;PATENT DEPARTMENT, 2600 HILLTOP DRIVE, P.O. BOX 4099, RICHMOND, CA, 94804-0099, US

(21) Appl. No.: 10/498,237

(22) PCT Filed: Dec. 10, 2002
(86) PCT No.: PCT/EP02/14010

(30) Foreign Application Priority Data
Dec. 12, 2001 (DE) 10162114.0

(51) Int. Cl. C07D 279/12; C07D 265/30; C07D 263/02; C07D 211/26

(52) U.S. Cl. 544/59; 544/162; 544/399; 546/231; 548/200; 548/215; 548/335.1

(57) ABSTRACT

Amidine derivatives of formula I, their tautomeric and isomeric forms and salts 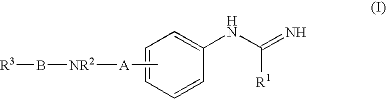 as well as the process for their production and their use for the production of a pharmaceutical agent are described.

The invention relates to amidine derivatives, the process for their production and their use for the production of a pharmaceutical agent for treating a disease that is triggered by nitrogen monoxide synthases.

In human cells, at least 3 forms of nitrogen monoxide synthases that convert arginine into nitrogen monoxide (NO) and citrulline exist. Two constitutive NO synthases (NOS) were identified that are present as calcium/calmodulin-dependent enzymes in the brain (ncNOS or NOS 1) or in the endothelium (ecNOS or NOS 3). Another isoform is the inducible NOS (iNOS or NOS 2) that is a virtually Ca⁺⁺-independent enzyme and is induced by endotoxin or other substances after activation of different cells.

NOS inhibitors and in particular selective inhibitors of NOS 1, NOS 2 or NOS 3 are therefore suitable for therapy of different diseases that are induced or aggravated by pathological concentrations of NO in cells. A number of reviews provide information on the action and inhibitors of NO synthases. There are mentioned, for example: Drugs 1, 321 (1998), or Current Pharmac. Design 3, 447 (1997).

As NOS inhibitors, different compounds, such as, for example, arginine derivatives, aminopyridines, cyclic amidine derivatives, phenylimidazoles and 3-amino-2H-1,4-benzoxazines and -benzothiazines are described. It is known from WO 95/05363 that open-chain amidines also show NOS-inhibitory action.

It has now been found that the amidines that are substituted according to the invention with halogenated alkyl have advantages compared to known compounds and can be used better than pharmaceutical agents.

The invention relates to compounds of formula I, their tautomeric and isomeric forms and salts 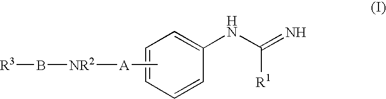
in which
A means —CH₂—, —(CH₂)₂—, —CH(CH₃)—,
R¹ means phenyl or 5- or 6-membered heteroaryl with 1-3 oxygen, sulfur or nitrogen atoms, whereby the phenyl radical and the heteroaryl radical can be substituted with halogen, C₁₋₄-alkyl, C₁₋₄-alkoxy or CF₃,
R² means hydrogen, C₁₋₄-alkyl, COOC₁₋₄-alkyl or COC₁₋₄-alkyl,
B means straight-chain or branched alkylene with 1-8 C atoms, which is substituted with halogen,
R³ means hydrogen, halogen, phenyl or 5- or 6-membered heteroaryl with 1-3 oxygen, sulfur or nitrogen atoms, whereby the phenyl radical and the heteroaryl radical can be substituted with halogen, C₁₋₄-alkyl, C₁₋₄-alkoxy or CF₃.

The compounds of formula I can be present in tautomeric, enantiomeric or diastereomeric form. The invention comprises all possible isomers, such as S- and R-enantiomers, diastereomers, racemates and mixtures thereof including the tautomeric compounds of formulas Ia and Ib

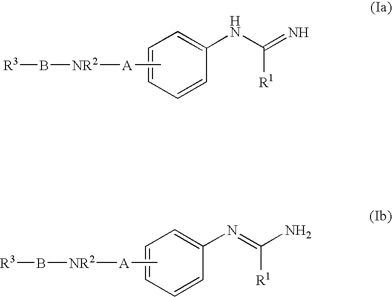

The physiologically compatible salts can be formed with inorganic and organic acids, such as, for example, oxalic acid, lactic acid, citric acid, fumaric acid, acetic acid, maleic acid, tartaric acid, phosphoric acid, HCl, HBr, sulfuric acid, p-toluenesulfonic acid, methanesulfonic acid, i.a.

In each case, alkyl means a straight-chain or branched alkyl group, such as, e.g., methyl, ethyl, propyl, isopropyl, n-butyl, sec. butyl, or tert. butyl.

As heteroaryl radicals that can be bonded via the heteroatom or a carbon atom, for example, the following 5- and 6-ring heteroaromatic compounds can be mentioned: imidazole, indole, isooxazole, isothiazole, furan, oxadiazole, oxazole, pyrazine, pyridazine, pyrimidine, pyridine, pyrazole, pyrrole, thiazole, triazole, thiophene, thiadiazole, benzimidazole, benzofuran, benzoxazole, isoquinoline, quinoline, 2-CH₃-3-amino-2H-1,4-benzoxazine.

Preferred heteroaryl groups for R¹ and R³ are: 2-furyl, 3-furyl, 2-pyridyl, 3-pyridyl, 4-pyridyl, 2-thienyl, and 3-thienyl. 2-Thienyl is especially preferred.

Phenyls and heteroaryls can in each case be substituted in one to three places in the same way or differently; the unsubstituted form is preferred.

In each case, halogen means fluorine, chlorine, bromine or iodine. For B, fluorine can be mentioned as the preferred halogen.

Straight-chain or branched C₁₋₈-alkylene is defined as methylene, ethylene, propylene, butylene, pentylene, etc., 1-methylethylene, 1-ethylethylene, 1-methylpropylene, 2-methylpropylene, 1-methylbutylene, 2-methylbutylene, 1-ethylbutylene, 2-ethylbutylene, 1-methylpentylene, 2-methylpentylene, 3-methylpentylene, etc., which is substituted in one to several places, preferably one to three places, with halogen, in particular with fluorine.

For example, fluoromethylene, 2-fluoroethylene, 1-fluoroprop-2-ylene, 4,4,4,-trifluorobutylene, 4,4,4-trifluorobut-2-ylene, 2,2,3,3,3-pentafluoropropylene, 2,2,2-trifluoroethylene, and 2,2,3,3,4,4,4-heptafluorobutylene can be mentioned.

The substituent R³—B—NR²-A- preferably stands in para- or meta-position for the amidine substituent.

If R³ means halogen, in particular fluorine is meant.

The invention also relates to the use of the compounds according to the invention for the production of a pharmaceutical agent for treating diseases that are produced by the action of nitrogen monoxide in pathological concentrations. These include neurodegenerative diseases, inflammatory diseases, autoimmune diseases, and cardiovascular diseases.

For example, there can be mentioned:

Cerebral ischemia, hypoxia and other neurodegenerative diseases that are associated with inflammations, such as multiple sclerosis, amyotropic lateral sclerosis and comparable sclerotic diseases, Parkinson's disease, Huntington's disease, Korksakoffs disease, epilepsy, vomiting, sleep disturbances, schizophrenia, depression, stress, pain, migraine, hypoglycemia, and dementia, such as, e.g., Alzheimer's disease, HIV dementia and presenile dementia.

They are also suitable for the treatment of diseases of the cardiovascular system and for the treatment of autoimmune and/or inflammatory diseases, such as hypotension, ARDS (adult respiratory distress syndrome), sepsis or septic shock, rheumatoid arthritis, osteoarthritis, insulin-dependent diabetes mellitus (IDDM), inflammatory disease of the pelvis/intestine (bowel disease), meningitis, glomerulonephritis, acute and chronic liver diseases, diseases by rejection (for example, allogenic heart, kidney or liver transplants) or inflammatory skin diseases such as psoriasis, etc.

Based on their profile of action, the compounds according to the invention are very well suited for inhibition of the neuronal NOS and for production of a pharmaceutical agent for treating CNS diseases.

To use the compounds according to the invention as pharmaceutical agents, the latter are brought into the form of a pharmaceutical preparation, which in addition to the active ingredient for enteral or parenteral administration contains suitable vehicles, adjuvants and/or additives. The administration can be carried out orally or sublingually as a solid in the form of capsules or tablets or as a liquid in the form of solutions, suspensions, elixirs, aerosols or emulsions or rectally in the form of suppositories or in the form of injection solutions that can optionally also be administered subcutaneously, intramuscularly or intravenously, or topically or intrathecally. As adjuvants for the desired pharmaceutical agent formulation, the inert organic and inorganic vehicles that are known to one skilled in the art are suitable, such as, e.g., water, gelatin, gum arabic, lactose, starch, magnesium stearate, talc, vegetable oils, polyalkylene glycols, etc. Moreover, preservatives, stabilizers, wetting agents, emulsifiers or salts for changing the osmotic pressure or buffers optionally can be contained.

For parenteral application, in particular injection solutions or suspensions, in particular aqueous solutions of the active compounds in polyhydroxyethoxylated castor oil, are suitable.

As vehicle systems, surface-active adjuvants, such as salts of bile acids, or animal or plant phospholipids, but also mixtures thereof as well as liposomes or components thereof, can also be used.

For oral administration, in particular tablets, coated tablets or capsules with talc and/or hydrocarbon vehicles or binders, such as, for example, lactose, corn or potato starch, are suitable. The administration can also be carried out in liquid form, such as for example, as a juice, to which optionally a sweetener is added.

The dosage of the active ingredients can vary depending on the method of administration, age and weight of the patient, the type and severity of the disease to be treated and similar factors. The daily dose is 1-2000 mg, preferably 20-500 mg, whereby the dose can be given as an individual dose to be administered once or divided into two or more daily doses.

The NOS-inhibitory action of the compounds of formula I and physiologically compatible salts thereof can be determined according to the methods of Bredt und Snyder in Proc. Natl. Acad. Sci. USA 86, 9030 (1989).

The production of the compounds according to the invention is carried out in that a compound of formula II or salts thereof 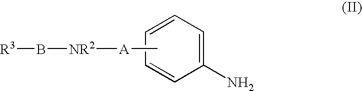
in which
A, B, R¹, R², and R³ have the above meaning, is reacted with a compound of formula III 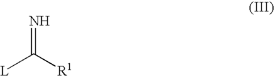
   in which R¹ has the above meaning and L represents a leaving group, whereby existing amino groups optionally are intermediately protected and optionally then alkylated, acylated, the isomers are separated, or the salts are formed.

The reaction of the compounds of formula II and formula III is performed in suitable solvents such as alcohols, for example methanol, ethanol, isopropanol or tert-butanol, whereby in general the reaction is carried out at the boiling point of the solvent. The reaction time depends on the type of solvent and the leaving group and can vary between a few hours and several days. By addition of an excess to tert-alkylamine, such as triethylamine, the reaction time can be accelerated. As leaving groups, common leaving groups are suitable, such as, for example, halide, alcoholates and thioalcoholates. As amino protective groups, for example, carbamates, such as tert-butoxycarbonyl, benzyloxycarbonyl or acetyl, are suitable. The cleavage of the protective group can be carried out with and without isolating the intermediate stage according to known processes.

If an alkylation of an amino group is desired, it can be alkylated according to commonly used methods, such as with alkyl halides. The acylation of the amino group is carried out in the usual way, for example with an acyl halide or acid anhydride in the presence of a base.

The isomer mixtures can be separated according to commonly used methods, such as, for example, crystallization, chromatography or salt formation, into enantiomers or E/Z-isomers. The enantiomers or enantiomer-pure diastereomers can also be obtained by chromatography on chiral phases, as well as by stereoselective syntheses.

The production of the salts is carried out in the usual way by a solution of the compound of formula 1-optionally also with protected amino groups—being mixed with the equivalent amount of or excess acid, which optionally is in solution, and the precipitate being separated or the solution being worked up in the usual way.

The compounds of formulas II and III are either known or can be produced according to methods that are known or described here.

For example, compounds of formula III can be obtained from the corresponding thiocarboxylic acid amides by reaction with methyl iodide (see, e.g.: B. Decroix et. al., Bull. Soc. Chim. Fr 1976, 621; K. Matsuda et al., Synth. Commun. 27, 2393 (1997)) or from the corresponding nitrites by reaction with alcohols and HCl (see, e.g.: Bercot-Vatteroni, Ann. Chim. (Paris) 7, 303 (1962); A. Couture et al., Synthesis 6, 456 (1989); R. A. Barcock et al., Tetrahedron 50, 4149 (1994); C. A. Veale et al., J. Med Chem. 38, 98 (1995)).

Compounds of formula II are obtained, for example, by reduction of the corresponding nitro compound to form amine (see, e.g., for surveys: M. Hudlicky, Reductions in Organic Chemistry, Ellis Horwood Limited, 1984; R. C. Larock, Comprehensive Organic Transformations, VCH Verlag, 1989). This reduction can be performed either catalytically in polar solvents at room temperature (see, e.g.: U. Hengartner et al., J. Org. Chem. 44, 3748 (1979)) or at elevated temperature under hydrogen pressure. As catalysts, metals such as Raney nickel or noble metal catalysts are suitable, such as, e.g., palladium, or platinum, optionally in the presence of barium sulfate. Instead of hydrogen, ammonium formate or formic acid can also be used in a known way. Under certain circumstances, however, complex metal hydrides can also be used, optionally in the presence of heavy metal salts. In principle, the reduction with zinc and ammonium chloride in water-ethanol-tetrahydrofuran mixtures is also possible. Another method is the reduction with dithionite as a reducing agent (Houben-Weyl, Vol. XI/1, p. 437). Another variant consists in the reduction of the nitro group with indium in ethanol with the addition of ammonium chloride (C. J. Moody, M. R. Pitts, Synlett 1998, 1028).

The intermediate compounds can be further processed as enantiomers, diastereomers, racemates or mixtures thereof.

Below, the production of a few precursors, intermediate products and products is described by way of example:

### EXAMPLE 1

### N-(3-{[(1R,2R)-2-Fluoro-1-methyl-2-phenylethyl]aminomethyl}phenyl)-thiophene-2-carboximidamide, Dihydrochloride

### a) 1-[(1R,2S)-2-Hydroxy-1-methyl-2-phenylethyl]-2,5-dimethyl-1H-pyrrole

15 g (99.206 mmol) of (1S, 2R)-(+)-norephedrine is refluxed with 11.3 g (99.206 mmol) of hexane-2,5-dione in 60 ml of methanol for four hours. After dilution with ethyl acetate, the organic phase is washed twice with saturated sodium bicarbonate solution and once with saturated common salt solution. After drying and spinning-in, the residue is chromatographed on silica gel (mobile solvent: ethyl acetate/hexane). 19.82 g (87%) of the desired compound is isolated.

MS (CI) m/e (relative intensity) 230 (M⁺, 100), 122 (10)

### b) 1-[(1R,2R)-2-Fluoro-1-methyl-2-phenylethyl]-2,5-dimethyl-1H-pyrrole

19.82 g (86.433 mmol) of 1-[(1R,2S)-2-hydroxy-1-methyl-2-phenylethyl]-2,5-dimethyl-1H-pyrrole and 38.27 ml (259.30 mmol) of DBU are dissolved in 1500 ml of toluene. After 39.25 g (129.818 mmol) of perfluorobutanesulfonic acid fluoride is added (heating to 30° C.), the batch is stirred for four hours at room temperature. The DBU is separated in a separatory funnel, and the organic phase is spun in until the dry state is reached. After chromatography on silica gel (mobile solvent: ethyl acetate/hexane), 5.35 g (27%) of the desired compound is obtained.

MS (CI) m/e (relative intensity) 232 (M⁺, 100), 212 (70.5), 122 (5.5)

### c) (1R,2R)-1-Fluoro-1-phenylpropane-2-amine

5.35 g (23.13 mmol) of 1-[(1R,2R)-2-fluoro-1-methyl-2-phenylethyl]-2,5-dimethyl-1H-pyrrole, 16.12 g (231.3 mmol) of hydroxylamine hydrochloride, 8.04 g (143.458 mmol) of potassium hydroxide, 108 ml of ethanol and 42 ml of water are refluxed overnight. The ethanol is spun off, and the residue is brought to a pH of 2 with 2 M hydrochloric acid. After extraction with methyl-tert-butyl ether is carried out four times, the aqueous phase is brought to a pH of 9 with potassium hydroxide and extracted three times with ethyl acetate. The combined ethyl acetate extracts are dried, and the solvent is spun off. 3.22 mg (91%) of the desired compound remains.

MS (CI) m/e (relative intensity) 154 (M⁺, 25.5), 145 (100)

### d) [(1R,2R)-2-Fluoro-1-methyl-2-phenylethyl](3-nitrobenzyl)amine

1.61 g (10.51 mmol) of (1R,2R)-1-fluoro-1-phenylpropane-2-amine and 1.59 g (10.51 mmol) of 3-nitrobenzaldehyde are dissolved in 64 ml of a mixture that consists of methanol and tetrahydrofuran (4:1). After stirring overnight, 215.33 mg (5.69 mmol) of sodium borohydride is added, and it is stirred for another hour. The batch is added to water and extracted four times with ethyl acetate. The combined organic phases are washed with saturated sodium chloride solution, dried and spun in. After chromatography on silica gel (mobile solvent: ethyl acetate/hexane), however, the desired compound is not obtained, but rather the corresponding imine (partially still contaminated with 3-nitrobenzaldehyde). This imine is dissolved again in methanol, mixed with additional sodium borohydride and stirred for two hours at 50° C. After repeated addition of sodium borohydride and additional stirring for two hours at 50° C., the batch is added to water and extracted three times with ethyl acetate. The combined organic extracts are dried, and the solvent is spun off After chromatography on silica gel (mobile solvent: ethyl acetate/hexane), 536.2 mg (65%) of the desired compound is obtained.

MS (CI) m/e (relative intensity) 289 (M⁺, 100), 179 (50)

### e) tert-Butyl-N-[(1R,2R)-2-fluoro-1-methyl-2-phenylethyl]-N-[3-nitrobenzyl]carbamate

536.2 g (1.86 mmol) of [(1R,2R)-2-fluoro-1-methyl-2-phenylethyl](3-nitrobenzyl)amine is stirred with 405 mg (1.86 mmol) of di-tert-butyl-dicarbonate and 0.38 ml (3.72 mmol) of triethylamine in 45 ml of dichloromethane for three days at 40° C. The batch is spun in until a dry state is reached, and the residue is chromatographed on silica gel (mobile solvent: ethyl acetate/hexane). 620 mg (86%) of the desired compound is isolated.

MS (CI) m/e (relative intensity) 389 (M⁺, 8), 350 (100), 179 (70)

### f) tert-Butyl-N-[(1R,2R)-2-fluoro-1-methyl-2-phenylethyl]-N-[3-aminobenzyl]carbamate

620 mg (1.596 mmol) of tert-butyl-N-[(1R,2R)-2-fluoro-1-methyl-2-phenylethyl]-N-[3-nitrobenzyl]carbamate is mixed in 42 ml of ethanol with 1.42 g (12.346 mmol) of indium and 4.2 ml of saturated ammonium chloride solution and refluxed for five hours. After cooling, it is diluted with methyl-tert-butyl ether, filtered on a glass-fiber filter and shaken with water and then saturated sodium chloride solution. After drying, the solvent is spun off, and the residue is chromatographed on silica gel (mobile solvent: ethyl acetate/hexane). 321 mg (56%) of the desired compound is obtained.

MS (CI) m/e (relative intensity) 359 (M⁺, 39), 283 (74), 149 (100); 106 (39)

### g) tert-Butyl-N-[(1R,2R)-2-fluoro-1-methyl-2-phenethyl]-N-[3-(2-thienylcarbamimidoyl)benzyl]carbamate

292 mg (0.817 mmol) of tert-butyl-N-[(1R,2R)-2-fluoro-1-methyl-2-phenylethyl]-N-[3-aminobenzyl]carbamate is refluxed with 141 mg (0.898 mmol) of methyl-thiophene-2-carboximidothioate for seven days in 35 ml of isopropanol. The solvent is spun off, and the residue is chromatographed on silica gel (mobile solvent: ethyl acetate/hexane). 63.7 mg (17%) of the desired compound is isolated.

### h) N-(3-{[(1R,2R)-2-Fluoro-1-methyl-2-phenylethyl]aminomethyl}phenyl)-thiophene-2-carboximidamide, Dihydrochloride

63.7 mg (0.136 mmol) of tert-butyl-N-[(1R,2R)-2-fluoro-1-methyl-2-phenethyl]-N-[3-(2-thienylcarbamimidoyl)benzyl]carbamate is dissolved in 2 ml of dioxane and mixed with two milliliters of a 4 M solution of hydrochloric acid in dioxane. After stirring overnight, the batch is spun in until a dry state is reached, and the residue is absorptively precipitated with a mixture that consists of dichloromethane/methyl-tert-butyl ether. The precipitated product is suctioned off: the yield is 31.7 mg (53%).

MS (CI) m/e (relative intensity) 368 (M⁺, 60), 348 (100), 258 (24); 132 (71)

The following are synthesized analogously with use of the corresponding starting materials:
N-(4-{[(1R,2R)-2-Fluoro-1-methyl-2-phenylethyl]aminomethyl}phenyl)-thiophene-2-carboximidamide, Dihydrochloride
N-(4-{[(1S,2S)-2-Fluoro-1-methyl-2-phenylethyl]aminomethyl}phenyl)-thiophene-2-carboximidamide, Dihydrochloride

### EXAMPLE 2

### N-(3-{[(RS)-3,3,3-Trifluoro-1-methylpropyl]aminomethyl}phenyl)-thiophene-2-carboximidamide, Dihydrochloride

### a) [(RS)-3,3,3-Trifluoro-1-methylpropyl]-(3-nitrobenzyl)amine

623 mg (3.81 mmol) of (RS)-3,3,3-trifluoro-1-methylpropylamine, hydrochloride, produced according to K.-R. Gassen and W. Kirmse, Chem. Ber. 119, 2233 (1986), is added in a mixture that consists of methanol and tetrahydrofuran (4:1). After the addition of 0.69 ml (4.95 mmol) of triethylamine, it is stirred for one-half hour at room temperature. Then, 576 mg (3.81 mmol) of 3-nitrobenzaldehyde is added, and it is stirred for five hours at 50° C. and overnight at room temperature. After the addition of 79 mg (2.09 mmol) of sodium borohydride, the batch is stirred for two hours at room temperature, for three hours at 50° C. and then overnight again at room temperature. The batch is added to water and extracted three times with ethyl acetate. The combined organic phases are washed with saturated sodium chloride solution, dried, and the solvent is spun off. After chromatography on silica gel (mobile solvent: ethyl acetate/hexane), 372.4 mg (37%) of the desired compound is obtained.

MS (EI) m/e (relative intensity) 262 (M⁺, 3), 179 (66), 136 (100)

### b) tert-Butyl-N-[(RS)-3,3,3-trifluoro-1-methylpropyl]-N-[3-nitrobenzyl]carbamate

364 mg (1.39 mmol) of [(RS)-3,3,3-trifluoro-1-methylpropyl]-(3-nitrobenzyl)amine is stirred with 333.92 mg (1.53 mmol) of di-tert.-butyl dicarbonate and 281.31 mg (2.78 mmol) of triethylamine in 28 ml of dichloromethane for 16 hours at room temperature. The batch is spun in until a dry state is reached, and the residue is put on a column on silica gel (mobile solvent: ethyl acetate/hexane). 275 mg (55%) of the desired compound is isolated.

MS (EI) m/e (relative intensity) 262 (M⁺-Boc, 3)179 (23), 134 (32), 57 (100)

### c) tert-Butyl-N-[(RS)-3,3,3-trifluoro-1-methylpropyl]-N-[3-amino-benzyl]carbamate

270 mg (0.745 mmol) of tert-butyl-N-[(RS)-3,3,3-trifluoro-1-methylpropyl]-N-[3-nitro-benzyl]carbamate is refluxed in 20 ml of ethanol with 663 mg (5.76 mmol) of indium and two milliliters of saturated ammonium chloride solution for five hours. After cooling, it is diluted with diethyl ether, filtered on a glass-fiber filter and washed with water and brine. After the solvent is dried and spun in, the residue is chromatographed on silica gel (mobile solvent: ethyl acetate/hexane). 104.2 mg (42%) of the desired compound is isolated.

MS (EI) m/e (relative intensity) 332 (M⁺, 7), 276 (30), 107 (100)

### d) 52 mg (0.12 mmol) of tert-Butyl-N-[(RS)-3,3,3-trifluoro-1-methylpropyl]-N-[3-(2-thienylcarbamimidoyl)benzyl]carbamate

174 mg (0.523 mmol), 52 mg (0.12 mmol) of tert-butyl-N-[(RS)-3,3,3-trifluoro-1-methylpropyl]-N-[3-amino-benzyl]carbamate were refluxed in 24 ml of isopropanol with 91 mg (0.58 mmol) of methyl-thiophene-2-carboximidothioate for seven days. The solvent is drawn off, and the residue is chromatographed on silica gel (mobile solvent: ethyl acetate/hexane). The desired compound is obtained in a 23% yield (54 mg).

MS (EI) m/e (relative intensity) 441 (M⁺, 8), 110 (42), 57 (100)

### e) N-(3-{[(RS)-3,3,3-Trifluoro-1-methylpropyl]aminomethyl}phenyl)thiophene-2-carboximidamide, Dihydrochloride

52 mg (0.12 mmol) of tert-butyl-N-[(RS)-3,3,3-trifluoro-1-methylpropyl]-N-[3-(2-thienylcarbamimidoyl)benzyl]carbamate is introduced into 1.8 ml of tetrahydrofuran and mixed with 0.8 ml of a 4 M solution of hydrochloric acid in dioxane. After stirring overnight at room temperature, it is mixed with two milliliters of ethyl acetate. After 10 minutes, the solid is suctioned off, washed with a little cold ethyl acetate and dried at 60° C. in a vacuum. 39.1 mg (80%) of the desired compound is isolated.

MS (EI) m/e (relative intensity) 341 (M⁺, 12), 216 (100), 199 (88)

The following is synthesized analogously with use of the corresponding starting materials:
N-(4-{[(RS)-3,3,3-Trifluoro-1-methylpropyl]aminomethyl}phenyl)thiophene-2-carboximidamide, Dihydrochloride

1) Amidine derivatives of general formula I, their tautomeric and isomeric forms and salts 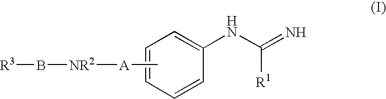
in which
A means —CH₂—, —(CH₂)₂—, —CH(CH₃)—,
R¹ means phenyl or 5- or 6-membered heteroaryl with 1-3 oxygen, sulfur or nitrogen atoms, whereby the phenyl radical and the heteroaryl radical can be substituted with halogen, C₁₋₄-alkyl, C₁₋₄-alkoxy or CF₃,
R² means hydrogen, C₁₋₄-alkyl, COOC₁₋₄-alkyl or COC₁₋₄-alkyl,
B means straight-chain or branched alkylene with 1-8 C atoms, which is substituted with halogen,
R³ means hydrogen, halogen, phenyl or 5- or 6-membered heteroaryl with 1-3 oxygen, sulfur or nitrogen atoms, whereby the phenyl radical and the heteroaryl radical can be substituted with halogen, C₁₋₄-alkyl, C₁₋₄-alkoxy or CF₃.

2. Amidine derivatives according to claim 1, characterized in that B means straight-chain or branched C₁₋₈-alkylene that is substituted with fluorine.

3. N-(3-{[(1R,2R)-2-Fluoro-1-methyl-2-phenylethyl]aminomethyl}phenyl)-thiophene-2-carboximidamide
N-(4-{[(1R,2R)-2-Fluoro-1-methyl-2-phenylethyl]aminomethyl}phenyl)-thiophene-2-carboximidamide
N-(4-{[(1S,2S)-2-Fluoro-1-methyl-2-phenylethyl]aminomethyl}phenyl)-thiophene-2-carboximidamide
N-(3-{[(RS)-3,3,3-Trifluoro-1-methylpropyl]aminomethyl}phenyl)thiophene-2-carboximidamide
N-(4-{[(RS)-3,3,3-Trifluoro-1-methylpropyl]aminomethyl}phenyl)thiophene-2-carboximidamide

4. Use of a compound according to claim 1 or 2 for the production of a pharmaceutical agent for treating a disease that is triggered by nitrogen monoxide synthase.

5. Use according to claim 4, wherein a disease of the central nervous system is treated.

6. Pharmaceutical agent that contains a compound according to claim 1 or 2 and one or more vehicles.

7. Process for the production of a compound of general formula I according to claim 1 or 2, wherein a compound of formula II or salts thereof 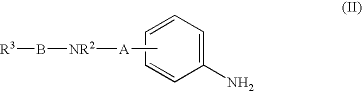
in which
A, B, R¹, R², and R³ have the above meaning, is reacted with a compound of 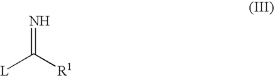
in which R¹ has the above meaning and L represents a leaving group, whereby existing amino groups optionally are intermediately protected and optionally then alkylated, acylated, the isomers are separated, or the salts are formed.
